(19) United States
(12) Patent Application Publication
(10) Pub. No.: US 2022/0325241 A1
(43) Pub. Date: Oct. 13, 2022

(54) IMMUNE EFFECTOR CELL FOR CO-EXPRESSING CHEMOKINE RECEPTOR

(71) Applicant: CRAGE MEDICAL CO., LIMITED, Hong Kong (CN)

(72) Inventors: Zonghai LI, Shanghai (CN); Ruixin SUN, Shanghai (CN)

(73) Assignee: CRAGE MEDICAL CO., LIMITED, Hong Kong (CN)

(21) Appl. No.: 17/634,050

(22) PCT Filed: Aug. 10, 2020
(86) PCT No.: PCT/CN2020/108265

§ 371 Date: Feb. 9, 2022

(30) Foreign Application Priority Data
Aug. 9, 2019 (CN) 201910735367.0

(51) Int. Cl. C12N 5/0783; C07K 14/725; C07K 14/715; C07K 16/24; C12N 15/62; C12N 15/85

(52) U.S. Cl. CPC C12N 5/0636; C07K 14/7051; C07K 14/7158; C07K 16/24; C12N 15/625; C12N 15/85; C12N 2800/107

(57) ABSTRACT

Provided are an immune effector cell for co-expressing a chemokine receptor, a pharmaceutical composition, a kit, and a method for treating a tumor. The immune effector cell comprises a receptor that specifically recognizes claudin 18.2 and a protein that recognizes SDF-1. Further provided are an expression construct, an expression vector, and a virus. The expression construct comprises an expression of a receptor that binds to a tumor-associated antigen and an expression of the protein that recognizes SDF-1, which are connected in sequence.

This application claims the priority of CN2019107353670, priority date: 2019/08/09.

### FIELD OF THE INVENTION

The invention belongs to the field of cellular immunotherapy. More specifically, the present invention relates to immune effector cells for co-expressing chemokine receptors and tumor-associated antigen-binding receptors.

### BACKGROUND OF THE INVENTION

Tumors, especially solid tumors, are a complex composed of tumor cells and their surrounding stromal cells and non-cell components. The occurrence and development of tumors are dynamic processes in which tumor cells and their microenvironment promote each other and co-evolve. The tumor microenvironment plays a vital role in the process of growth and metastasis of tumors. Cancer associated fibroblasts (CAFs), as one of the most important components in the tumor microenvironment, mainly secrete various extracellular matrix components (such as collagen, proteoglycans, proteases, growth factors, cell factors and chemokines) to promote tumor development.

In the field of immune cell therapy, the homing of immune effector cells to tumor sites is an important prerequisite for immune cells to play a role in killing tumors, and it is also a difficult problem in this field. For example, pancreatic cancer, one of the most lethal malignant tumors. Because pancreatic tumor cells can activate tumor-specific immune responses, at the same time, they can stimulate immunosuppressive responses at a higher intensity and establish an immunosuppressive microenvironment to evade immune surveillance, traditional radiotherapy and chemotherapy regimens are not effective in the treatment of pancreatic cancer. Even with cellular immunotherapy which has a good development momentum at present, in the treatment of pancreatic cancer, due to the limited homing ability of therapeutic cells, it also encounters a relatively greater challenge.

Therefore, further research is needed in this field to find ways to better homing immune cells to tumor sites, and to exert their killing effect on tumors, so as to improve the efficacy of immune effector cells for tumor immunotherapy.

### SUMMARY OF THE INVENTION

The first aspect of the present application relates to an immune effector cell for co-expressing a chemokine receptor, wherein the cell comprises: a receptor specifically recognizing claudin 18.2; and a protein recognizing SDF-1.

Specifically, the protein recognizing SDF-1 is an antibody recognizing SDF-1 or a receptor for SDF-1.

Specifically, the receptor for SDF-1 is CXCR4 or CXCR7.

Specifically, an amino acid sequence of scFV of the receptor specifically recognizing claudin 18.2 has at least 90% identity with the amino acid sequence shown in SEQ ID NO: 2.

Specifically, the immune effector cell is selected from the group consisting of: a T cell, a NK cell, a NKT cell, a macrophage, a CIK cell, and an immune effector cell derived from a stem cell.

Specifically, the receptor for SDF-1 is CXCR4, and preferably, an amino acid sequence of CXCR4 has at least 90% identity with the sequence shown in SEQ ID NO: 19. Preferably, the amino acid sequence of CXCR4 is the sequence shown in SEQ ID NO: 19.

Specifically, the receptor specifically recognizing claudin 18.2 is a chimeric receptor; preferably, the chimeric receptor is selected from the group consisting of: a chimeric antigen receptor (CAR), a chimeric T cell receptor, or a T cell antigen coupler (TAC).

Specifically, the chimeric receptor is a chimeric antigen receptor, and the chimeric antigen receptor comprises an extracellular domain, a transmembrane region, and an intracellular signaling region that are sequentially connected; wherein the extracellular domain comprises an antibody or a ligand; preferably, the antibody is a single chain antibody or a domain antibody. The transmembrane region is a sequence comprising a transmembrane region of CD8 or CD28. The intracellular signaling region is selected from: sequences of intracellular signaling regions of CD3ζ, FcεRIγ, CD27, CD28, CD137, CD134, or a combination thereof.

Preferably, the extracellular domain of the chimeric antigen receptor is scFv, and further preferably, the scFv comprises HDCR1, HCDR2, HCDR3 shown in SEQ ID NOs: 24, 25, and 26, and LDCR1, LCDR2, LCDR3 shown in SEQ ID NOs: 27, 28, and 29. More preferably, the extracellular domain of the chimeric antigen receptor comprises the amino acid sequence shown in SEQ ID NO:2.

In a specific embodiment, the chimeric antigen receptor comprises any of the following (i), (ii) and (iii):

(i) The aforementioned scFv, CD8 and CD3ζ;

(ii) The aforementioned scFv, CD8, CD137 and CD3ζ; or

(iii) The aforementioned scFv, the transmembrane region of the CD28 molecule, the intracellular signaling region of the CD28 molecule, and CD3ζ.

In a specific embodiment, the cell comprises a nucleic acid having at least 90% identity with the nucleotide sequence shown in SEQ ID NO:18. Preferably, the cell comprises the nucleotide sequence shown in SEQ ID NO:18.

In a specific embodiment, the chimeric antigen receptor has at least 90% identity with the amino acid sequences shown in SEQ ID NO: 21, 22 or 23.

The second aspect of the present application relates to an immune effector cell for co-expressing a chemokine receptor, wherein the cell comprises: a receptor specifically recognizing pancreatic cancer antigen; and a protein recognizing SDF-1.

Specifically, the protein recognizing SDF-1 is an antibody recognizing SDF-1, or a receptor for SDF-1;

Specifically, the receptor for SDF-1 is CXCR4 or CXCR7.

Specifically, an amino acid sequence of scFV of a receptor specifically recognizing claudin 18.2 has at least 90% identity with the amino acid sequence shown in SEQ ID NO: 2.

Specifically, the immune effector cell is selected from the group consisting of: a T cell, a NK cell, a NKT cell, a macrophage, a CIK cell, and an immune effector cells derived from a stem cell.

Specifically, the receptor for SDF-1 is CXCR4, and preferably, an amino acid sequence of CXCR4 has at least 90% identity with the sequence shown in SEQ ID NO: 19. Preferably, the amino acid sequence of CXCR4 is the sequence shown in SEQ ID NO: 19.

Specifically, the receptor specifically recognizing a pancreatic cancer antigen is a chimeric receptor; preferably, the chimeric receptor is selected from the group consisting of: a chimeric antigen receptor (CAR), a chimeric T cell receptor, or a T cell antigen coupler (TAC).

Specifically, the chimeric receptor is a chimeric antigen receptor, and the chimeric antigen receptor comprises an extracellular domain, a transmembrane region, and an intracellular signaling region that are sequentially connected; wherein the extracellular domain comprises an antibody or a ligand; preferably, the antibody is a single chain antibody or a domain antibody. The transmembrane region is a sequence comprising a transmembrane region of CD8 or CD28. The intracellular signaling region is selected from sequences of intracellular signaling regions of CD3ζ, FcεRIγ, CD27, CD28, CD137, CD134, or a combination thereof.

Preferably, the extracellular domain of the chimeric antigen receptor is scFv, and further preferably, the scFv comprises HDCR1, HCDR2, HCDR3 shown in SEQ ID NOs: 24, 25, and 26, and LDCR1, LCDR2, LCDR3 shown in SEQ ID NOs: 27, 28, and 29. More preferably, the extracellular domain of the chimeric antigen receptor comprises the amino acid sequence shown in SEQ ID NO:2.

In a specific embodiment, the chimeric antigen receptor comprises any of the following (i), (ii) and (iii):

(i) The aforementioned scFv, CD8 and CD3ζ;

(ii) The aforementioned scFv, CD8, CD137 and CD3ζ; or

(iii) The aforementioned scFv, the transmembrane region of the CD28 molecule, the intracellular signaling region of the CD28 molecule, and CD3ζ.

In a specific embodiment, the cell comprises a nucleic acid having at least 90% identity with the nucleotide sequence shown in SEQ ID NO:18. Preferably, the cell comprises the nucleotide sequence shown in SEQ ID NO:18.

In a specific embodiment, the chimeric antigen receptor has at least 90% identity with the amino acid sequences shown in SEQ ID NO: 21, 22 or 23.

In the third aspect of the present application, provided is an expression construct, wherein the expression construct comprises an expression of a tumor-associated antigen-binding receptor and an expression of a protein recognizing SDF-1, which are connected in sequence; preferably, the two expressions are connected with a tandem fragment; more preferably, the tandem fragment comprises F2A, PA2, T2A, and/or E2A.

Specifically, the protein recognizing SDF-1 is an antibody recognizing SDF-1, or a receptor for SDF-1; more preferably, the receptor for SDF-1 is CXCR4 or CXCR7.

More specifically, the receptor for SDF-1 is CXCR4.

Specifically, the tumor-associated antigen-binding receptor has at least 90% identity with the amino acid sequences shown in SEQ ID NO: 21, 22 or 23.

More specifically, the nucleic acid sequence of an expression of CXCR4 is a sequence having at least 90% identity with the nucleic acid sequence encoding the amino acid sequence shown in SEQ ID NO: 19.

In the fourth aspect of the present application, provided is an expression vector, which comprises the expression construct encoding the above-mentioned third aspect of the present application.

In the fifth aspect of the present application, provided is a virus, characterized in that, it comprises the vector described in the fourth aspect of the present application.

In the sixth aspect of the present application, provided is a pharmaceutical composition comprising the immune effector cell described in the above-mentioned first or second aspect of the present application; and a pharmaceutically acceptable carrier.

In the seventh aspect of the present application, provided is a medicine kit, comprising: a container, and the pharmaceutical composition of the sixth aspect of the present application in the container; or a container, and the immune effector cell described in the first aspect or the second aspect of the present application in the container.

In the eighth aspect of the present application, provided is a method for treating tumors, wherein the immune effector cell described in the first or second aspect of the present application is administered to an individual suffering from a tumor, and lymphocyte depletion is performed or not performed on the individual; preferably, lymphocyte depletion is performed on the individual.

### The Beneficial Effects of the Present Invention

The immune effector cell constructed in the present invention can interact with CXCL12 in the tumor microenvironment, so that the immune effector cell homes to tumor cells through chemotaxis under the action of CXCL12, and the immune effector cell can better play the role of killing tumors. This is because CAR-T cells with high expression of CXCR4 can spread or transfer to tumor tissues with high expression of SDF1α/CXCL12 through chemotaxis. CXCR4 on the cell surface is activated after binding to its ligand. The activated CXCR4 can participate in cell proliferation, cell migration and other processes through multiple signal pathways, such as Wnt/β-catenin, NF-κB, etc.

### BRIEF DESCRIPTION OF THE DRAWINGS

FIG. 1A shows the plasmid map of MSCV-CLDN18.2-BBZ; FIG. 1B shows the plasmid map of MSCV-CLDN18.2-BBZ-F2A-CXCR4;

FIG. 2 shows the positive rate of CAR-T cell infection;

FIG. 3 shows the in vitro killing toxicity of CLDN18.2-BBZ CAR T cells and CLDN18.2-BBZ-CXCR4 CAR T cells to tumor cells;

FIG. 4 shows the in vivo treatment experiment of CAR-T cells on the subcutaneous xenograft mouse model of PANC02-A2 pancreatic cancer cell after lymphocyte depletion: FIG. 4A shows the measurement results of the transplanted tumor volumes, and FIG. 4B shows the body weight measurement results of the mice. FIG. 4C shows the weight measurement results of the transplanted tumors, and FIG. 4D shows the results of inhibition of PANC02-A2 pancreatic cancer by CLDN18.2-BBZ and CLDN18.2-BBZ-CXCR4 CAR-T cells.

### DETAIL DESCRIPTION OF THE INVENTION

After in-depth research, the present inventors revealed for the first time a genetically engineered immune effector cell that co-expresses CXCR4. The immune effector cell can interact with CXCL12 in the tumor microenvironment, so that the immune effector cell can home to tumor cells through chemotaxis under the action of CXCL12, and the immune effector cell can better play the role of killing tumors.

Unless specifically defined, all technical and scientific terms used herein have the same meanings commonly understood by those skilled in the fields of gene therapy, biochemistry, genetics, and molecular biology. Methods and materials similar or equivalent to those described herein can be used in the implementation of the present invention. All publications, patent applications, patents and other references mentioned herein are incorporated herein by reference in their entirety. In case of conflict, the present specification, including definitions, will control. In addition, unless otherwise specified, the materials, methods, and examples are illustrative only and not intended to be limitation.

Unless otherwise specified, the traditional techniques of cell biology, cell culture, molecular biology, transgenic biology, microbiology, recombinant DNA and immunology used in the implementation of the present invention all fall within the technical scope of the art. These techniques are fully explained in the literatures, for example, Current Protocols in Molecular Biology (FrederickM.AUSUBEL, 2000, Wileyand sonInc, Library of Congress, USA); Molecular Cloning: A Laboratory Manual, Third Edition, (Sambrooketal, 2001, Cold Spring Harbor, NewYork: Cold Spring Harbor Laboratory Press); Oligonucleotide Synthesis (M. J. Gaited., 1984); Mullis et al. U.S. Pat. No. 4,683,195; Nucleic Acid Hybridization (B. D. Harries & S. J. Higginseds. 1984); Transcription and Translation (B. D. Hames & S. J. Higginseds. 1984); Culture Of Animal Cells (R. I. Freshney, Alan R. Liss, Inc., 1987);Immobilized Cells And Enzymes (IRL Press, 1986);B. Perbal, A Practical Guide To Molecular Cloning (1984);the series, Methods In ENZYMOLOGY (J. Abelson and M. Simon, eds.-in-chief, Academic Press, Inc., New York), “Gene Expression Technology” (D. Goeddel, ed.);Gene Transfer Vectors For Mammalian Cells (J. H. Miller and M. P. Caloseds., 1987, Cold Spring Harbor Laboratory);Immunochemical Methods In Cell And Molecular Biology (Mayer and Walker, eds., Academic Press, London, 1987); Hand book Of Experimental Immunology, volume I-IV (D. M. Weir and C. C. Blackwell, eds., 1986); and Manipulating the Mouse Embryo (Cold Spring Harbor Laboratory Press, Cold Spring Harbor, N.Y., 1986).

In order to better understand the present invention, related terms are defined as follows:

The term “genetically engineered cell” refers to a cell modified by means of genetic engineering. Specifically, the genetically engineered cell of the present invention refers to a cell co-expressing a receptor that binds to an antigen (such as a tumor antigen) and an exogenous open reading frame (for example, CXCR4 open reading frame), so that it can exert targeted killing effect; especially refers to a T cell co-expressing a chimeric antigen receptor which specifically binds to tumor antigens and CXCR4.

The term “CXCR4” is a specific receptor for chemokine stromal cell-derived factor-1 (CXCL12). CXCR4 is expressed in some cells and organs. Its coding gene is located on human chromosome 2q21, which encodes 352 amino acid residues. Its coding sequence is highly conserved. Its α-helix spans the membrane seven times, has an extracellular N-terminus, three extracellular loops, three intracellular loops and an intracellular C-terminus, wherein the N-terminus is a main binding site of a ligand. The C-terminus is located in a cytoplasm, it has Ser/Thr sites, and has nothing to do with the binding of receptors and ligands, and is directly involved in signaling.

The term “CXCL12” (also known as SDF-1), belonging to the CXC chemokine family, is a chemotactic protein secreted by bone marrow stromal cells and other closely related mesothelial and epithelial cells. Its gene is located on the long arm of chromosome 10. A coding region of the gene is 267 bp in length, which encodes 89 amino acid polypeptides and produces six splice variants. CXCL12 is expressed in stromal fibroblasts of brain, breast, liver, lung and other tissues and organs. It participates in cell migration and leukocyte chemotaxis, and plays an important role in tumor proliferation and migration. CXCL12 can bind to a tumor cell surface receptor CXCR4. An axis formed by the specific binding of CXCL12/CXCR4 can not only promote tumor growth, tumor angiogenesis, but also promote tumor cell proliferation and migration.

The term “immune effector cell” refers to a cell that participates in an immune response, for example, promoting immune effects. Examples of immune effector cells include T cells, for example, α/β T cells and γ/δ T cells, B cells, natural killer (NK) cells, natural killer T (NKT) cells, mast cells, and bone marrow-derived phagocytes. Preferably, the T cells include autologous T cells, xenogeneic T cells, and allogeneic T cells, and the natural killer cells are allogeneic NK cells. As used herein, the term “immune effector function or immune effector response” refers to functions or responses of an immune effector cell that, for example, enhances or promotes immune attack on target cells. For example, immune effector function or response refers to the properties of T cells or NK cells that promote the killing of target cells or the inhibition of growth or proliferation.

The term “chimeric antigen receptor” (CAR) comprises an extracellular antigen binding domain, a transmembrane domain, and an intracellular signaling domain. Intracellular signaling domains include functional signaling domains of stimulatory molecules and/or costimulatory molecules. In one aspect, the stimulatory molecule is the (chain that binds to the T cell receptor complex; in one aspect, the cytoplasmic signaling domain further comprises one or more functional signaling domains of costimulatory molecules, such as 4-1BB (i.e., CD137), CD27, and/or CD28.

The term “extracellular binding domain” (is also referred to as extracellular antigen binding domain) includes antibodies or ligands that specifically recognize antigens (such as tumor-associated antigens), and the antibodies are preferably single-chain antibodies or domain antibodies. More preferably, the extracellular antigen binding domain of the chimeric antigen receptor is connected to the transmembrane domain of CD8 or CD28 with a CD8 hinge region, and the transmembrane domain is immediately followed by the intracellular signal domain.

The term “transmembrane domain” (also referred to as a transmembrane region) refers to the region of a protein sequence that spans the cell membrane and may include one or more additional amino acids adjacent to the transmembrane domain, such as one or more amino acids associated with the extracellular region of the protein from which the transmembrane protein is derived (for example, 1, 2, 3, 4, 5, 6, 7, 8, 9, 10 up to 15 amino acids in the extracellular region) and/or one or more additional amino acids associated with the extracellular region of the protein from which the transmembrane protein is derived (e.g., 1, 2, 3, 4, 5, 6, 7, 8, 9, 10 up to 15 amino acids in the intracellular region). In one aspect, the transmembrane domain is a domain related to one of the other domains of the chimeric receptor, for example, in one embodiment, the transmembrane domain may be from the same protein from which the signal transduction domain, the co-stimulator domain or the hinge domain is derived. In some cases, transmembrane domains can be selectively modified or modified by amino acid substitutions to prevent such domains from binding to transmembrane domains of the same or different surface membrane proteins, for example, to minimize its interaction with the other members of the receptor complex. In one aspect, the transmembrane domain can homodimerize with another chimeric receptor on the surface of the cell expressing the chimeric receptor. The transmembrane domain can be derived from natural or recombinant sources. When the source is natural, the domain can be derived from any membrane-bound protein or transmembrane protein. In one aspect, the transmembrane domain can transmit signals to the intracellular domain whenever the chimeric receptor binds to the target. The transmembrane domains particularly used in the present invention may include at least the following transmembrane domains: for example, α, β or ζ chains of T-cell receptors, CD28, CD27, CD3e, CD45, CD4, CD5, CD8, CD9, CD16, CD22, CD33, CD37, CD64, CD80, CD86, CD134, CD137, CD154. In certain embodiments, the transmembrane domains may include at least the following transmembrane domains: for example KIRDS2, OX40, CD2, CD27, LFA-1(CD11a, CD18), ICOS(CD278), 4-1BB(CD137), GITR, CD40, BAFFR, HVEM(LIGHTR), SLAMF7, NKp80(KLRF1), NKp44, NKp30, NKp46, CD160, CD19, IL2β, IL2Ry, IL7Ra, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1(CD226), SLAMF4(CD244, 2B4), CD84, CD96(Tactile), CEACAM1, CRTAM, Ly9(CD229), CD160(BY55), PSGL1, CD100(SEMA4D), SLAMF6(NTB-A, Ly108), SLAM(SLAMF1, CD150, IPO-3), BLAME(SLAMF8), SELPLG(CD162), LTBR, PAG/Cbp, NKG2D, NKG2C.

In some cases, the transmembrane domain may be connected to the extracellular region of the CAR, such as the antigen binding domain of the CAR, via a hinge (for example, a hinge from a human protein). Optionally, a short oligopeptide or polypeptide linker between 2 to 10 amino acids in length can form a bond between the transmembrane domain of the CAR and the cytoplasmic region. The glycine-serine dimer is provided as a particularly suitable linker.

The term “intracellular domain” and “intracellular signal domain” have the same meaning, including intracellular signaling domain. The intracellular signaling domain refers to the part of the protein that transduces the immune effector function signal and leads the cell to perform a specific function, and can lead the activation of the immune effector function of the immune cell. The immune effector function of T cells can be, for example, cytolytic activity or auxiliary activity, including secretion of cytokines. Although the entire intracellular signaling domain can usually be used, in many cases it is not necessary to use the entire chain. As long as the immune effector function signal can be transduced, a truncated part can be used instead of the entire chain.

Those skilled in the art can also use technical means known in the art to promote the fusion expression of chemokine receptors and chimeric antigen receptors on the surface of immune effector cells, including but not limited to the use of self-cleaving sequences for the fusion expression of chemokine receptors and chimeric antigen receptors. In a specific embodiment, the cleavable protein can be selected from but not limited to 2A polypeptides and IRES. 2A polypeptides can be selected from F2A, PA2, T2A, E2A, etc.; the self-cleaving sequence is preferably F2A or P2A. Wherein, F2A is a core sequence of 2A (or “self-cleaving polypeptide 2A”) derived from foot-and-mouth disease virus. It has the “self-cleaving” function of 2A and can realize the co-expression of upstream and downstream genes. 2A provides an effective and feasible strategy for the construction of polycistronic vectors for gene therapy due to its high cleaving efficiency, high balance of upstream and downstream gene expression, and short self-sequence.

The term “tumor-associated antigen” refers to an antigen expressed in a tumor. The “tumor-associated antigen” can be selected from (but not limited to): EGFR, GPC3, HER2, EphA2, Claudin 18.1, Claudin 18.2, Claudin 6, GD2, EpCAM, mesothelin, CD19, CD20, ASGPR1, EGFRvIII, de4 EGFR, CD19, CD33, IL13R, LMP1, PLAC1, NY-ESO-1, MAGE4, MUC1, MUC16, LeY, CEA, CAIX (carbonic anhydrase IX), CD123.

In the present invention, a variety of tumors known in the art can be included in the present invention. For example, the tumors include (but are not limited to): breast cancer, liver cancer, gastric cancer, colorectal cancer, ovarian cancer, lung cancer, pancreas cancer.

The term “claudin 18.2” or “claudin 18A2” (CLD18.2, CLD18A2, CLDN18A2, or CLDN18.2) herein can also refer to homologs, orthologues, and orthologs, codon optimized form, truncated form, fragmented form, mutant form or any other known derivative form, such as post-translational modification variant, of the known claudin 18A2 sequences. In some embodiments, the claudin 18A2 or claudin 18A2 peptide is a peptide with GenBank accession number NP_001002026 (mRNA: NM_001002026)

The term “CAFs”, is also referred to as tumor-associated fibroblasts, is the most abundant host cell in the microenvironment of solid tumors, and acquires an activated phenotype under the action of the microenvironment. Different from normal fibroblasts, CAFs are characterized by the expression of a-smooth muscle actin (a-SMA) and fibroblast activation protein (FAP), which can secrete a large number of growth factors, such as VEGF, TGF-β, hepatocyte growth factor, etc. It can synthesize and deposit ECM, and produce various collagens, adhesion proteins, and mediate ECM remodeling. CAFs have been verified to be important in the occurrence and development of, and the metastasis and recurrence of tumors, and it has been revealed that they promote tumor growth by dominating the tumor microenvironment.

The term “antibody” refers to a protein or polypeptide sequence derived from an immunoglobulin molecule that specifically binds to an antigen. Antibodies can be polyclonal or monoclonal, multi-chain or single-chain, intact immunoglobulin or antibody fragments, and can be derived from natural sources or recombinant sources. An antibody can be a tetramer of immunoglobulin molecules.

“Single-chain antibody (scFv)” herein refers to an antibody defined as follows, it is a recombinant protein comprising a heavy chain variable region (VH) and a light chain variable region (VL) connected by a linker. The linker associates the two domains to ultimately form the antigen binding site. The size of scFv is generally ⅙ of that of a complete antibody. The single-chain antibody is preferably an amino acid chain sequence encoded by a nucleotide chain. The single-chain antibody used in the present invention can be further modified by using conventional techniques known in the art alone or in combination, such as amino acid deletion, insertion, substitution, addition, and/or recombination and/or other modification methods. The method of introducing such modifications into the DNA sequence of an antibody based on its amino acid sequence is well known to those skilled in the art; see, for example, Sambrook, Molecular Cloning: Laboratory Manual, Cold Spring Harbor Laboratory (2002) N.Y. The modifications referred to are preferably performed at the nucleic acid level. The aforementioned single-chain antibody may also include derivatives thereof.

The term “single domain antibody” (sdAb), also known as a nanobody, consists of a single variable domain of an antibody. Single-domain antibodies have small molecular weight and strong stability. Although their structure is simple, they can still achieve a binding affinity to specific antigens that is comparable to or even higher than that of traditional antibodies. Therefore, single domain antibodies are widely used in bispecific antibodies and cell therapy (such as chimeric antigen receptor T cells).

The term “chimeric receptor” refers to a fusion molecule formed by linking DNA fragments or cDNAs corresponding to proteins from different sources using gene recombination technology, and includes extracellular domains, transmembrane domains and intracellular domains. Chimeric receptors include but are not limited to: a chimeric antigen receptor (CAR), a chimeric T cell receptor (TCR), and a T cell antigen coupler (TAC).

The term “T cell receptor (TCR)” mediates the recognition of specific major histocompatibility complex (MHC)-restricted peptide antigens by T cells, including classic TCR receptors and optimized TCR receptors. The classic TCR receptor is composed of two peptide chains, α and β. Each peptide chain can be divided into variable region (V region), constant region (C region), transmembrane region and cytoplasmic region, etc., and its antigen specificity exists in V regions, and the V regions (Vα, Vβ) each have three hypervariable regions CDR1, CDR2, and CDR3. In one aspect, for T cells expressing classic TCR, methods such as antigen stimulation on T cells can be used to induce the specificity of the TCR of T cells to the target antigen.

The term “T cell antigen coupler (TAC)” includes three functional domains: 1. an antigen binding domain, including single-chain antibodies, and designed ankyrin repeat protein (DARPin) or other targeting groups; 2. an extracellular domain, a single-chain antibody that binds to CD3, so an to bring TAC receptors into proximity with TCR receptors; 3. a transmembrane region and an intracellular region of the CD4 co-receptor, wherein the intracellular region is connected to the protein kinase LCK to catalyze the phosphorylation of immunoreceptor tyrosine activation motifs (ITAMs) of the TCR complex as the initial step of T cell activation.

The term “chimeric T cell receptor” includes recombinant polypeptides derived from various polypeptides constituting the TCR. It can bind to the surface antigens of target cells and interact with other polypeptides of the complete TCR complex, and they are usually co-localized on T cell surface. The chimeric T cell receptor is composed of a TCR subunit and an antigen binding domain composed of a human or humanized antibody domain. Wherein, the TCR subunit includes at least a part of the TCR extracellular domain, transmembrane domain, and the stimulation domain of the intracellular signal domain of TCR intracellular domain; the TCR subunit and the antibody domain are operatively connected, wherein the extracellular, transmembrane, and intracellular signal domains of the TCR subunit are derived from CD3 ε or CD3γ, and, the chimeric T cell receptor is integrated into the TCR expressed on the T cell.

The term “signaling domain” refers to a functional part of a protein that functions by transmitting information in a cell, and is used to regulate the cell activity through a definite signaling pathway by generating a second messenger or acting as an effector in response to such a messenger. The intracellular signaling domain can include all intracellular parts of the molecule, or a full natural intracellular signaling domain, or functional fragments or derivatives thereof.

The term “co-stimulatory molecule” refers to a signal that binds to a cell stimulating signal molecule, such as TCR/CD3, and in combination leads to the proliferation of T cells and/or the up-regulation or down-regulation of key molecules.

The terms “stimulation” and “activation” are used interchangeably and can refer to the process by which a cell changes from a resting state to an active state. The process can include a response to antigens, migration, and/or phenotypic or genetic changes in functional activity status. For example, the term “activation” can refer to the process of gradual activation of T cells. For example, T cells may require at least one signal to be fully activated.

The genetically engineered cells of the present invention can be used to prepare pharmaceutical compositions or diagnostic reagents. In addition to including an effective amount of the immune cells, the composition may also include a pharmaceutically acceptable carrier. The term “pharmaceutically acceptable” means that when the molecular entities and compositions are properly administered to animals or humans, they will not produce adverse, allergic or other adverse reactions, such as cell cryoprotectants. The term “cell cryoprotectant” can be a composition, for example, it can contain isotonic salt, buffer salt, glycerin, DMSO, ethylene glycol, propylene glycol, acetamide, polyvinylpyrrolidone (PVP), sucrose, poly ethylene glycol, dextran, albumin, hydroxyethyl starch, serum, etc.

The composition of the present invention can be made into various dosage forms according to needs, and the physicians can determine the beneficial dosage for the patient according to factors such as the patient's type, age, weight, general disease condition, and administration method. The administration method can be injection or other treatment methods.

The term “lymphocyte depletion” or “lymphocyte cleansing” refers to the removal of lymphocytes from the subject in the body. This includes administration of lymphocyte scavengers, whole body radiation therapy, or a combination thereof. For example, in order to increase the amplification or maintenance of immune effector cells in the subject, before, at the same time as, or after administering a therapeutically effective amount of CAR-T cells or any combination thereof, the subject can be administered with one or more drugs that can greatly deplete the lymphocytes in the subject, whole body radiation therapy, or a combination thereof, either alone or in combination.

Lymphocyte depletion treatment can be given under conditions sufficient to achieve a lymphocyte clearance rate of 50%-100% in the subject.

The lymphocyte scavenger may be an anti-tumor chemotherapeutic agent, such as fludarabine, cyclophosphamide, or a combination thereof. The doctor can choose the specific lymphocyte scavenger and its suitable dosage according to the subject to be treated, such as CAMPATH, anti-CD3 antibody, cyclosporin, FK506, rapamycin, mycophenolic acid, steroid, FR901228, Melphalan, cyclophosphamide, fludarabine and whole body radiation therapy.

The immune effector cells administration is administered before, during, and after the lymphocyte depletion treatment, and can also be administered in combination, that is, before and during, before and after, during and after, or before, during and after the lymphocyte depletion treatment. In certain embodiments, the lymphocyte depletion treatment is administered 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 month or any combination thereof before the immune effector cell therapy. In certain embodiments, the lymphocyte depletion treatment is administered 1 hour, 2 hours, 3 hours, 4 hours, 5 hours, 6 hours, 7 hours, 8 hours, 9 hours, 10 hours, 11 hours, 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 month or any combination thereof after the administration of immune effector cell therapy.

The present invention will be further explained below in conjunction with specific examples. It should be understood that these examples are only used to illustrate the present invention and not to limit the scope of the present invention. The experimental methods without specific conditions in the following examples usually follow the conventional conditions as described in J. Sambrook et al., Molecular Cloning: A Laboratory Manual, Third Edition, Science Press, 2002, or follow the conditions suggested by the manufacturers.

### EXAMPLE

### Example 1. Genetically Engineered Cell Construction

1. Plasmid Construction

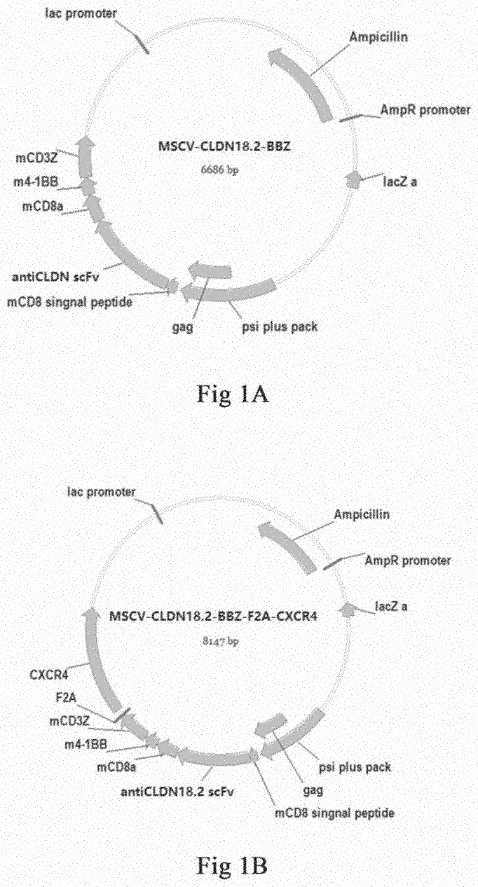

Using conventional molecular biology methods in the art, the scFv used in this example is a Claudin 18.2-targeted antibody (the nucleotide sequence is shown in SEQ ID NO: 1 and the amino acid sequence is shown in SEQ ID NO: 2), with HDCR1, HCDR2, HCDR3 shown in SEQ ID NOs: 24, 25, and 26, and LDCR1, LCDR2, LCDR3 shown in SEQ ID NOs: 27, 28, and 29. The chimeric antigen receptor adopted is the second-generation chimeric antigen receptor, with the transmembrane domain of CD8, the intracellular domain of 4-1BB (CD137), and CD3ζ. Referring to the plasmid map shown in FIG. 1A, the plasmid MSCV-CLDN18.2-BBZ is constructed.

In order to verify the results in mice, the transmembrane and intracellular domains selected for the preparation of CAR in this example are all derived from mice. If the present invention is used for clinical purposes, the transmembrane domain and intracellular domain for the preparation of CAR are preferably from human sources.

Using MSCV-IRES-GFP (purchased from addgene) as a vector, a retroviral plasmid MSCV-CLDN18.2-BBZ expressing the second-generation chimeric antigen receptor is constructed. CLDN18.2-BBZ sequence consists of mouse CD8a signal peptide (nucleotide sequence is shown in SEQ ID NO: 3, amino acid sequence is shown in SEQ ID NO: 4), scFv targeting Claudin 18.2 (nucleotide sequence is shown in SEQ ID NO: 1, amino acid sequence is shown in SEQ ID NO: 2), mouse CD8 hinge and transmembrane domain (nucleotide sequence is shown in SEQ ID NO: 5, amino acid sequence is shown in SEQ ID NO: 6), mouse 4-1BB intracellular signaling domain (nucleotide sequence is shown in SEQ ID NO: 7, amino acid sequence is shown in SEQ ID NO: 8), and CD3C, the intracellular segment of mouse CD3 (nucleotide sequence is shown in SEQ ID NO: 9, amino acid sequence is shown in SEQ ID NO: 10).

F2A-CXCR4 sequence is inserted on the basis of MSCV-CLDN18.2-BBZ plasmid to construct a lentiviral plasmid MSCV-CLDN18.2-BBz-CXCR4 expressing the second-generation chimeric antigen receptor. The plasmid map is shown in FIG. 1B. F2A-CXCR4 is composed of F2A (nucleotide sequence is shown in SEQ ID NO: 11, amino acid sequence is shown in SEQ ID NO: 12), mouse CXCR4 (nucleotide sequence is shown in SEQ ID NO: 13, amino acid sequence is shown in SEQ ID NO: 14).

2. Using conventional virus packaging methods in the art, such as the methods described in Di S, Zhou M, Pan Z, Sun R, Chen M, Jiang H, et al. Combined Adjuvant of Poly I: C Improves Antitumor Effects of CAR-T Cells. 2019). Frontiers in oncology. 9:241, the MSCV-CLDN18.2-BBZ and MSCV-CLDN18.2-BBz-CXCR4 plasmids are separately transfected into 293T to package retrovirus, and retroviruses are obtained.

3. T cell activation: Grind the spleen of C57BL/6 mice to obtain lymphocytes. After treatment with the CD3+ mouse T cell negative screening kit (Stemcell), the obtained mouse CD3+T lymphocytes are stimulated to be activated by adding Dynabeads Mouse T-activator CD3/CD28 magnetic beads at a volume ratio of 1:1. They are put in a cell culture incubator. The medium is RPMI 1640 complete medium (10% FBS+50 μM β-mercaptoethanol+100U/mL IL-2+ Ing/mL IL-7).

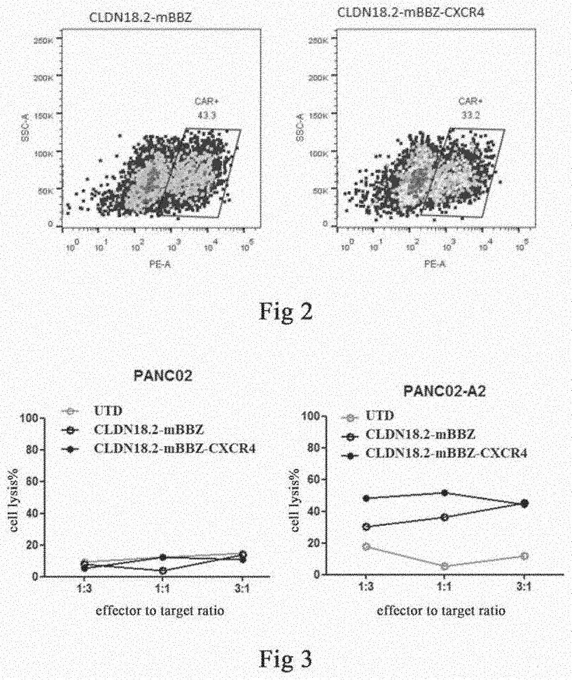

CD3+T lymphocytes from mouse spleens that have been activated for 24 hours are inoculated into a 24-well plate coated with Retronectin (5 μg/mL). After 24 hours of infection with retrovirus, it is replaced with fresh medium, and mouse CLDN18.2-BBZ cells and CLDN18.2-BBZ-CXCR4 cells are obtained. The infection positive rate of CAR-T cells is detected by flow cytometry. As shown in FIG. 2, the infection positive rate of MSCV-CLDN18.2-BBZ cells is 43.3%, and the infection positive rate of MSCV-CLDN18.2-BBZ-CXCR4 cells is 33.2%.

### Example 2. In Vitro Killing Toxicity Test of CAR-T on Mouse Pancreatic Cells PANC02-A2

2.1 Construction of mouse pancreatic cancer cell PANC02-A2 stably overexpressing the extracellular domain of huClaudin 18.2

In the mouse pancreatic cancer cell line PANC02 (purchased from ATCC), the full-length sequence of CLDN18.2 (amino acid sequence is shown in SEQ ID NO: 20) is overexpressed by lentiviral vector to obtain expression-stable PANC02-A2 cell line; PANC02-A2 positive cell line is screened with flow sorting technology, and the cell line is used to carry out the follow-up research, PANC02 cells are used as negative control cells for follow-up experiments.

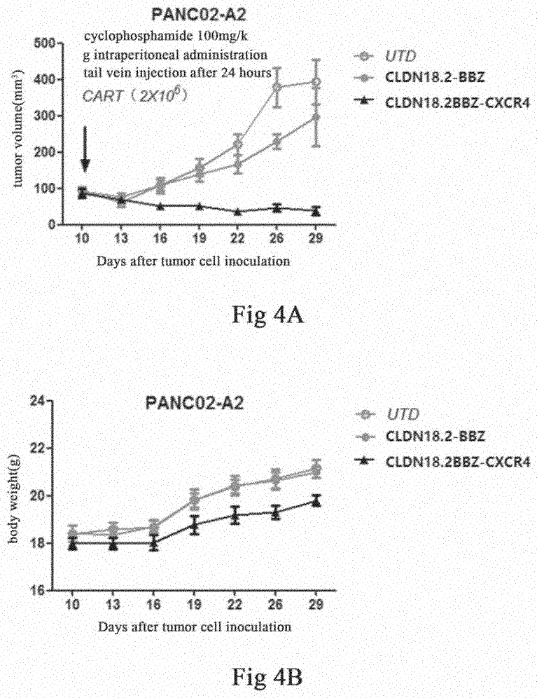

2.2 The untreated mouse T cells (UTD) and CLDN18.2-BBZ CAR T cells in Example 1 (the nucleotide sequence of CLDN18.2-BBZ is shown in SEQ ID NO: 15, the amino acid sequence is shown in SEQ ID NO: 16) and CLDN18.2-BBZ-CXCR4 CAR T cells (the nucleotide sequence of CLDN18.2-BBZ-CXCR4 is shown in SEQ ID NO: 17, the amino acid sequence is shown in SEQ ID NO: 18) are separately co-incubated with PANC02 cells and PANC02-A2 cells at 1:3, 1:1, 3:1. After 16 hours, Cytox 96 Non-Radioactive Cytotoxicity Assay is used to detect the secretion of LDH in the supernatant, and the killing toxicity of CLDN18.2-BBZ CAR T cells and CLDN18.2-BBZ-CXCR4 CAR T cells to tumor cells are calculated (as shown in FIG. 3). Specific detection steps and calculation methods is referred to the manual of Promaga Cytox 96 Non-Radioactive Cytotoxicity Assay (Promaga, REF: G1782). It can be seen from FIG. 3 that when the effector to target ratio is 1:3 and 1:1, the killing effect of CLDN18.2-BBZ-CXCR4 CAR T on tumors is better than that of CLDN18.2-BBZ CAR T cells.

### Example 3. Anti-Tumor Treatment Test of Subcutaneous Xenograft

(1) Establishment and grouping of subcutaneous xenograft models of mouse pancreatic cancer:

The well-growing PANC02-A2 cells in the logarithmic growth phase are collected, and 1×10⁶ cells are subcutaneously inoculated into C57BL/6 mice (mice with normal immune system). The day of tumor cell inoculation is recorded as day 0 (i.e., Day0).

(2) Cyclophosphamide is administered to mice by intraperitoneal injection on the 10th day (Day 10) after tumor inoculation. Dosage of cyclophosphamide: 100 mg/kg. 0.2 g of cyclophosphamide powder is fully dissolved in 20 ml of normal saline, and 200 μl of which is injected intraperitoneally in each mouse.

(3) On the 11th day (Day11) after tumor inoculation, CAR T cells (2×10⁶) are injected through the tail vein. CLDN18.2-BBZ cells and CLDN18.2-BBZ-CXCR4 cells are constructed as described in step 1 of this example.

The mice are divided into 3 groups, 5 in each group:

UTD group: 2×10⁶ mouse T cells without virus infection are given;

CLDN18.2-BBZ group: 2×10⁶ CLDN18.2-BBZ-CAR-T cells are given;

CLDN18.2-BBZ-CXCR4 group: 2×10⁶ CLDN18.2-BBZ-CXCR4-CAR-T cells are given;

(4) Detection of tumor volume. The tumor volume changes in mice are consistently observed and measured, and recorded three times a week. The tumor volume calculation formula is: tumor volume=(tumor length*tumor width²)/2.

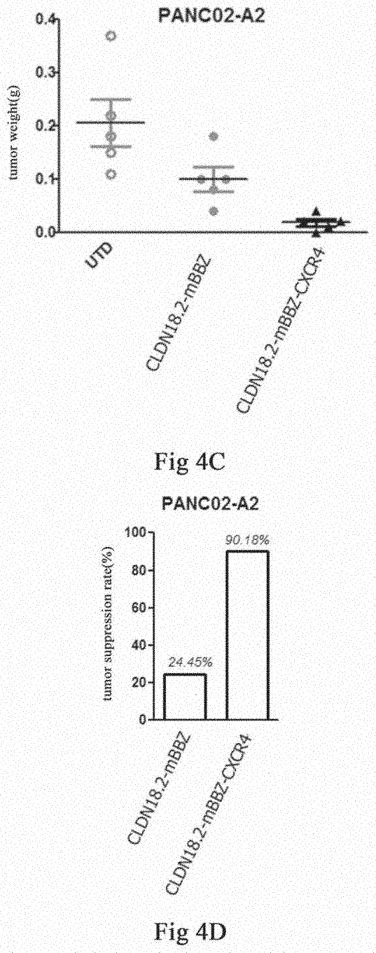

The results of mouse tumor volume detection are shown in FIG. 4A, and the results show that CART co-expressing CXCR4 can significantly inhibit mouse tumor volume. At the same time, it is detected that the body weight of the mice in each group did not change significantly (as shown in FIG. 4B), suggesting that CART co-expressing CXCR4 did not cause significant toxic effects on mice.

(5) Measurement of tumor weight. On Day 29, the mice are euthanized, the mouse tumors are stripped and the tumor weights are weighed. The specific statistical results are shown in FIG. 4C. It suggests that CAR-T cells co-expressing CXCR4 have a better anti-tumor effect on pancreatic cancer in mice.

(6) Tumor inhibition rate calculation. The values of the final tumor volumes of Day29 mice are taken for calculation. The calculation formula is tumor inhibition rate (%)=(the value of the final tumor volume of the mice in the UTD group—the value of the final tumor volume of the mice in the test group)/the value of the final tumor volume of the mice in the UTD group*100. As shown in FIG. 4D, the tumor inhibition rate in the CLDN18.2-BBZ group is 24.25%, which did not achieve a good effect of tumor growth inhibition; the tumor inhibition rate in the CLDN18.2-BBZ-CXCR4 group is 90.18%, suggesting that CAR-T cells co-expressing CXCR4 can inhibit the growth of pancreatic cancer in mice.

As an example, the antibodies used in the above examples are humanized antibodies, but it should be known that the antibodies used can be murine antibodies or humanized antibodies, and the adopted transmembrane domain and intracellular domain can also be of different species according to different purposes, such as human.

As an example, although that used in the above embodiments are CAR-T cells, the T cells can also express other cytokines that enhance the function of CAR-T cells, such as CAR-T cells co-expressing CAR and type I interferons, CAR-T cells co-expressing CAR and PD1, etc.

As an example, although that used in the above embodiments are CAR-T cells, other immune cells, such as NK cells, NK-T cells, and specific subtypes of immune cells, such as γ/δ T cells, etc. can also be selected.

As an example, the above embodiment selects a mouse-derived CAR, but its signal peptide, hinge region, transmembrane region, etc. can be selected from other species according to different purposes, including but not limited to human signal peptides, hinge regions, transmembrane domains and intracellular regions, for example, the adopted amino acid sequence of the CAR may be the amino acid sequences shown in SEQ ID NOs: 21, 22, and 23. Murine antibodies or humanized antibodies or fully human antibodies against different targets can also be selected as antibodies according to different purposes.

All documents mentioned in the present invention are cited as references in this application, as if each document is individually cited as a reference. In addition, it should be understood that after reading the above teaching content of the present invention, those skilled in the art can make various changes or modifications to the present invention, and these equivalent forms also fall within the scope defined by the claims of this application.

### Sequences Used Herein are as Follows:

| SEQ ID | | |
|---|---|---|
| NO. | Sequence name | Sequence |
| 1 | CLDN18.2-scFv | CAGGTGCAGCTGCAGGAGAGCGGCCCCGGCCTGATCAAGCCCAGCCAGA |
| | nucleotide sequence | CCCTGAGCCTGACCTGCACCGTGAGCGGCGGCAGCATCAGCAGCGGCTA |
| | | CAACTGGCACTGGATCCGGCAGCCCCCCGGCAAGGGCCTGGAGTGGATC |
| | | GGCTACATCCACTACACCGGCAGCACCAACTACAACCCCGCCCTGCGGA |
| | | GCCGGGTGACCATCAGCGTGGACACCAGCAAGAACCAGTTCAGCCTGAA |
| | | GCTGAGCAGCGTGACCGCCGCCGACACCGCCATCTACTACTGCGCCCGG |
| | | ATCTACAACGGCAACAGCTTCCCCTACTGGGGCCAGGGCACCACCGTGA |
| | | CCGTGAGCAGCGGTGGAGGCGGTTCAGGCGGAGGTGGTTCTGGCGGTGG |
| | | CGGATCGGACATCGTGATGACCCAGAGCCCCGACAGCCTGGCCGTGAGC |
| | | CTGGGCGAGCGGGCCACCATCAACTGCAAGAGCAGCCAGAGCCTGTTCA |
| | | ACAGCGGCAACCAGAAGAACTACCTGACCTGGTACCAGCAGAAGCCCGG |
| | | CCAGCCCCCCAAGCTGCTGATCTACTGGGCCAGCACCCGGGAGAGCGGC |
| | | GTGCCCGACCGGTTCAGCGGCAGCGGCAGCGGCACCGACTTCACCCTGA |
| | | CCATCAGCAGCCTGCAGGCCGAGGACGTGGCCGTGTACTACTGCCAGAA |
| | | CGCCTACAGCTTCCCCTACACCTTCGGCGGCGGCACCAAGCTGGAGATCA |
| | | AGCGG |
| 2 | CLDN18.2-scFv amino | QVQLQESGPGLIKPSQTLSLTCTVSGGSISSGYNWHWIRQPPGKGLEWIGYIH |
| | acid sequence | YTGSTNYNPALRSRVTISVDTSKNQFSLKLSSVTAADTAIYYCARIYNGNSFP |
| | | YWGQGTTVTVSSGGGGSGGGGSGGGGSDIVMTQSPDSLAVSLGERATINCK |
| | | SSQSLFNSGNQKNYLTWYQQKPGQPPKLLIYWASTRESGVPDRFSGSGSGTD |
| | | FTLTISSLQAEDVAVYYCQNAYSFPYTFGGGTKLEIKR |
| 3 | mouse CD8 α signal | ATGGCCTCACCGTTGACCCGCTTTCTGTCGCTGAACCTGCTGCTGCTGGGT |
| | peptide nucleotide | GAGTCGATTATCCTGGGGAGTGGAGAAGCT |
| | sequence | |
| 4 | mouse CD8 α signal | MASPLTRFLSLNLLLLGESIILGSGEA |
| | peptide amino acid | |
| | sequence | |
| 5 | Mouse CD8 hinge and | ACTACTACCAAGCCAGTGCTGCGAACTCCCTCACCTGTGCACCCTACCGG |
| | transmembrane domain | GACATCTCAGCCCCAGAGACCAGAAGATTGTCGGCCCCGTGGCTCAGTG |
| | nucleotide sequence | AAGGGGACCGGATTGGACTTCGCCTGTGATATTTACATCTGGGCACCCTT |
| | | GGCCGGAATCTGCGTGGCCCTTCTGCTGTCCTTGATCATCACTCTCATCTG |
| | | CTACCACAGGAGCCGA |
| 6 | Mouse CD8 hinge and | TTTKPVLRTPSPVHPTGTSQPQRPEDCRPRGSVKGTGLDFACDIYIWAPLAGI |
| | transmembrane domain | CVALLLSLIITLICYHRSR |
| | amino acid sequence | |
| 7 | Mouse 4-1BB | AAATGGATCAGGAAAAAATTCCCCCACATATTCAAGCAACCATTTAAGA |
| | intracellular signaling | AGACCACTGGAGCAGCTCAAGAGGAAGATGCTTGTAGCTGCCGATGTCC |
| | domain nucleotide | ACAGGAAGAAGAAGGAGGAGGAGGAGGCTATGAGCTG |
| | sequence | |
| 8 | Mouse 4-1BB | KWIRKKFPHIFKQPFKKTTGAAQEEDACSCRCPQEEEGGGGGYEL |
| | intracellular signaling | |
| | domain amino acid | |
| | sequence | |
| 9 | nucleotide sequence of | AGCAGGAGTGCAGAGACTGCTGCCAACCTGCAGGACCCCAACCAGCTCT |
| | CD3ζ, the intracellular | ACAATGAGCTCAATCTAGGGCGAAGAGAGGAATATGACGTCTTGGAGAA |
| | segment of mouse CD3 | GAAGCGGGCTCGGGATCCAGAGATGGGAGGCAAACAGCAGAGGAGGAG |
| | | GAACCCCCAGGAAGGCGTATACAATGCACTGCAGAAAGACAAGATGGCA |
| | | GAAGCCTACAGTGAGATCGGCACAAAAGGCGAGAGGCGGAGAGGCAAG |
| | | GGGCACGATGGCCTTTACCAGGGTCTCAGCACTGCCACCAAGGACACCT |
| | | ATGATGCCCTGCATATGCAGACCCTGGCC |
| 10 | amino acid sequence of | SRSAETAANLQDPNQLYNELNLGRREEYDVLEKKRARDPEMGGKQQRRRN |
| | CD3ζ, the intracellular | PQEGVYNALQKDKMAEAYSEIGTKGERRRGKGHDGLYQGLSTATKDTYDA |
| | segment of mouse CD3 | LHMQTLA |
| 11 | F2A nucleotide | GTGAAACAGACTTTGAATTTTGACCTTCTGAAGTTGGCAGGAGACGTTGA |
| | sequence | GTCCAACCCTGGGCCC |
| 12 | F2A amino acid | VKQTLNFDLLKLAGDVESNPGP |
| | sequence | |
| 13 | mouse CXCR4 | ATGGAACCGATCAGTATATACACTTCTGATAACTACTCTGAAGAAGTGGG |
| | nucleotide sequence | TTCTGGAGACTATGACTCCAACAAGGAACCCTGCTTCCGGGATGAAAAC |
| | | GTCCATTTCAATAGGATCTTCCTGCCCACCATCTACTTCATCATCTTCTTG |
| | | ACTGGCATAGTCGGCAATGGATTGGTGATCCTGGTCATGGGTTACCAGAA |
| | | GAAGCTAAGGAGCATGACGGACAAGTACCGGCTGCACCTGTCAGTGGCT |
| | | GACCTCCTCTTTGTCATCACACTCCCCTTCTGGGCAGTTGATGCCATGGCT |
| | | GACTGGTACTTTGGGAAATTTTTGTGTAAGGCTGTCCATATCATCTACACT |
| | | GTCAACCTCTACAGCAGCGTTCTCATCCTGGCCTTCATCAGCCTGGACCG |
| | | GTACCTCGCTATTGTCCACGCCACCAACAGTCAGAGGCCAAGGAAACTG |
| | | CTGGCTGAAAAGGCAGTCTATGTGGGCGTCTGGATCCCAGCCCTCCTCCT |
| | | GACTATACCTGACTTCATCTTTGCCGACGTCAGCCAGGGGGACATCAGTC |
| | | AGGGGGATGACAGGTACATCTGTGACCGCCTTTACCCCGATAGCCTGTGG |
| | | ATGGTGGTGTTTCAATTCCAGCATATAATGGTGGGTCTCGTCCTGCCCGG |
| | | CATCGTCATCCTCTCCTGTTACTGCATCATCATCTCTAAGCTGTCACACTC |
| | | CAAGGGCCACCAGAAGCGCAAGGCCCTCAAGACGACAGTCATCCTCATC |
| | | CTAGCTTTCTTTGCCTGCTGGCTGCCATATTATGTGGGGATCAGCATCGAC |
| | | TCCTTCATCCTTTTGGGGGTCATCAAGCAAGGATGTGACTTCGAGAGCAT |
| | | CGTGCACAAGTGGATCTCCATCACAGAGGCCCTCGCCTTCTTCCACTGTT |
| | | GCCTGAACCCCATCCTCTATGCCTTCCTCGGGGCCAAGTTCAAAAGCTCT |
| | | GCCCAGCATGCACTCAACTCCATGAGCAGAGGCTCCAGCCTCAAGATCCT |
| | | TTCCAAAGGAAAGCGGGGTGGACACTCTTCCGTCTCCACGGAGTCAGAA |
| | | TCCTCCAGTTTTCACTCCAGCTAA |
| 14 | mouse CXCR4 amino | MEPISIYTSDNYSEEVGSGDYDSNKEPCFRDENVHFNRIFLPTIYFIIFLTGIVG |
| | acid sequence | NGLVILVMGYQKKLRSMTDKYRLHLSVADLLFVITLPFWAVDAMADWYFG |
| | | KFLCKAVHIIYTVNLYSSVLILAFISLDRYLAIVHATNSQRPRKLLAEKAVYV |
| | | GVWIPALLLTIPDFIFADVSQGDISQGDDRYICDRLYPDSLWMVVFQFQHIMV |
| | | GLVLPGIVILSCYCIIISKLSHSKGHQKRKALKTTVILILAFFACWLPYYVGISI |
| | | DSFILLGVIKQGCDFESIVHKWISITEALAFFHCCLNPILYAFLGAKFKSSAQH |
| | | ALNSMSRGSSLKILSKGKRGGHSSVSTESESSSFHSS |
| 15 | CLDN18.2-BBZ | CAGGTGCAGCTGCAGGAGAGCGGCCCCGGCCTGATCAAGCCCAGCCAGA |
| | nucleotide sequence | CCCTGAGCCTGACCTGCACCGTGAGCGGCGGCAGCATCAGCAGCGGCTA |
| | | CAACTGGCACTGGATCCGGCAGCCCCCCGGCAAGGGCCTGGAGTGGATC |
| | | GGCTACATCCACTACACCGGCAGCACCAACTACAACCCCGCCCTGCGGA |
| | | GCCGGGTGACCATCAGCGTGGACACCAGCAAGAACCAGTTCAGCCTGAA |
| | | GCTGAGCAGCGTGACCGCCGCCGACACCGCCATCTACTACTGCGCCCGG |
| | | ATCTACAACGGCAACAGCTTCCCCTACTGGGGCCAGGGCACCACCGTGA |
| | | CCGTGAGCAGCGGTGGAGGCGGTTCAGGCGGAGGTGGTTCTGGCGGTGG |
| | | CGGATCGGACATCGTGATGACCCAGAGCCCCGACAGCCTGGCCGTGAGC |
| | | CTGGGCGAGCGGGCCACCATCAACTGCAAGAGCAGCCAGAGCCTGTTCA |
| | | ACAGCGGCAACCAGAAGAACTACCTGACCTGGTACCAGCAGAAGCCCGG |
| | | CCAGCCCCCCAAGCTGCTGATCTACTGGGCCAGCACCCGGGAGAGCGGC |
| | | GTGCCCGACCGGTTCAGCGGCAGCGGCAGCGGCACCGACTTCACCCTGA |
| | | CCATCAGCAGCCTGCAGGCCGAGGACGTGGCCGTGTACTACTGCCAGAA |
| | | CGCCTACAGCTTCCCCTACACCTTCGGCGGCGGCACCAAGCTGGAGATCA |
| | | AGCGGACTACTACCAAGCCAGTGCTGCGAACTCCCTCACCTGTGCACCCT |
| | | ACCGGGACATCTCAGCCCCAGAGACCAGAAGATTGTCGGCCCCGTGGCT |
| | | CAGTGAAGGGGACCGGATTGGACTTCGCCTGTGATATTTACATCTGGGCA |
| | | CCCTTGGCCGGAATCTGCGTGGCCCTTCTGCTGTCCTTGATCATCACTCTC |
| | | ATCTGCTACCACAGGAGCCGAAAATGGATCAGGAAAAAATTCCCCCACA |
| | | TATTCAAGCAACCATTTAAGAAGACCACTGGAGCAGCTCAAGAGGAAGA |
| | | TGCTTGTAGCTGCCGATGTCCACAGGAAGAAGAAGGAGGAGGAGGAGGC |
| | | TATGAGCTGAGCAGGAGTGCAGAGACTGCTGCCAACCTGCAGGACCCCA |
| | | ACCAGCTCTACAATGAGCTCAATCTAGGGCGAAGAGAGGAATATGACGT |
| | | CTTGGAGAAGAAGCGGGCTCGGGATCCAGAGATGGGAGGCAAACAGCA |
| | | GAGGAGGAGGAACCCCCAGGAAGGCGTATACAATGCACTGCAGAAAGA |
| | | CAAGATGGCAGAAGCCTACAGTGAGATCGGCACAAAAGGCGAGAGGCG |
| | | GAGAGGCAAGGGGCACGATGGCCTTTACCAGGGTCTCAGCACTGCCACC |
| | | AAGGACACCTATGATGCCCTGCATATGCAGACCCTGGCC |
| 16 | CLDN18.2-BBZ | QVQLQESGPGLIKPSQTLSLTCTVSGGSISSGYNWHWIRQPPGKGLEWIGYIH |
| | amino acid sequence | YTGSTNYNPALRSRVTISVDTSKNQFSLKLSSVTAADTAIYYCARIYNGNSFP |
| | | YWGQGTTVTVSSGGGGSGGGGSGGGGSDIVMTQSPDSLAVSLGERATINCK |
| | | SSQSLFNSGNQKNYLTWYQQKPGQPPKLLIYWASTRESGVPDRFSGSGSGTD |
| | | FTLTISSLQAEDVAVYYCQNAYSFPYTFGGGTKLEIKRTTTPAPRPPTPAPTIA |
| | | SQPLSLRPEACRPAAGGAVHTRGLDFACDIYIWAPLAGTCGVLLLSLVITLYC |
| | | KRGRKKLLYIFKQPFMRPVQTTQEEDGCSCRFPEEEEGGCELRVKFSRSADA |
| | | PAYQQGQNQLYNELNLGRREEYDVLDKRRGRDPEMGGKPQRRKNPQEGLY |
| | | NELQKDKMAEAYSEIGMKGERRRGKGHDGLYQGLSTATKDTYDALHMQA |
| | | LPPR |
| 17 | CLDN18.2-BBZ-CXCR | CAGGTGCAGCTGCAGGAGAGCGGCCCCGGCCTGATCAAGCCCAGCCAGA |
| | 4 nucleotide sequence | CCCTGAGCCTGACCTGCACCGTGAGCGGCGGCAGCATCAGCAGCGGCTA |
| | | CAACTGGCACTGGATCCGGCAGCCCCCCGGCAAGGGCCTGGAGTGGATC |
| | | GGCTACATCCACTACACCGGCAGCACCAACTACAACCCCGCCCTGCGGA |
| | | GCCGGGTGACCATCAGCGTGGACACCAGCAAGAACCAGTTCAGCCTGAA |
| | | GCTGAGCAGCGTGACCGCCGCCGACACCGCCATCTACTACTGCGCCCGG |
| | | ATCTACAACGGCAACAGCTTCCCCTACTGGGGCCAGGGCACCACCGTGA |
| | | CCGTGAGCAGCGGTGGAGGCGGTTCAGGCGGAGGTGGTTCTGGCGGTGG |
| | | CGGATCGGACATCGTGATGACCCAGAGCCCCGACAGCCTGGCCGTGAGC |
| | | CTGGGCGAGCGGGCCACCATCAACTGCAAGAGCAGCCAGAGCCTGTTCA |
| | | ACAGCGGCAACCAGAAGAACTACCTGACCTGGTACCAGCAGAAGCCCGG |
| | | CCAGCCCCCCAAGCTGCTGATCTACTGGGCCAGCACCCGGGAGAGCGGC |
| | | GTGCCCGACCGGTTCAGCGGCAGCGGCAGCGGCACCGACTTCACCCTGA |
| | | CCATCAGCAGCCTGCAGGCCGAGGACGTGGCCGTGTACTACTGCCAGAA |
| | | CGCCTACAGCTTCCCCTACACCTTCGGCGGCGGCACCAAGCTGGAGATCA |
| | | AGCGGACTACTACCAAGCCAGTGCTGCGAACTCCCTCACCTGTGCACCCT |
| | | ACCGGGACATCTCAGCCCCAGAGACCAGAAGATTGTCGGCCCCGTGGCT |
| | | CAGTGAAGGGGACCGGATTGGACTTCGCCTGTGATATTTACATCTGGGCA |
| | | CCCTTGGCCGGAATCTGCGTGGCCCTTCTGCTGTCCTTGATCATCACTCTC |
| | | ATCTGCTACCACAGGAGCCGAAAATGGATCAGGAAAAAATTCCCCCACA |
| | | TATTCAAGCAACCATTTAAGAAGACCACTGGAGCAGCTCAAGAGGAAGA |
| | | TGCTTGTAGCTGCCGATGTCCACAGGAAGAAGAAGGAGGAGGAGGAGGC |
| | | TATGAGCTGAGCAGGAGTGCAGAGACTGCTGCCAACCTGCAGGACCCCA |
| | | ACCAGCTCTACAATGAGCTCAATCTAGGGCGAAGAGAGGAATATGACGT |
| | | CTTGGAGAAGAAGCGGGCTCGGGATCCAGAGATGGGAGGCAAACAGCA |
| | | GAGGAGGAGGAACCCCCAGGAAGGCGTATACAATGCACTGCAGAAAGA |
| | | CAAGATGGCAGAAGCCTACAGTGAGATCGGCACAAAAGGCGAGAGGCG |
| | | GAGAGGCAAGGGGCACGATGGCCTTTACCAGGGTCTCAGCACTGCCACC |
| | | AAGGACACCTATGATGCCCTGCATATGCAGACCCTGGCCGTGAAACAGA |
| | | CTTTGAATTTTGACCTTCTGAAGTTGGCAGGAGACGTTGAGTCCAACCCT |
| | | GGGCCCATGGAACCGATCAGTATATACACTTCTGATAACTACTCTGAAGA |
| | | AGTGGGTTCTGGAGACTATGACTCCAACAAGGAACCCTGCTTCCGGGATG |
| | | AAAACGTCCATTTCAATAGGATCTTCCTGCCCACCATCTACTTCATCATCT |
| | | TCTTGACTGGCATAGTCGGCAATGGATTGGTGATCCTGGTCATGGGTTAC |
| | | CAGAAGAAGCTAAGGAGCATGACGGACAAGTACCGGCTGCACCTGTCAG |
| | | TGGCTGACCTCCTCTTTGTCATCACACTCCCCTTCTGGGCAGTTGATGCCA |
| | | TGGCTGACTGGTACTTTGGGAAATTTTTGTGTAAGGCTGTCCATATCATCT |
| | | ACACTGTCAACCTCTACAGCAGCGTTCTCATCCTGGCCTTCATCAGCCTG |
| | | GACCGGTACCTCGCTATTGTCCACGCCACCAACAGTCAGAGGCCAAGGA |
| | | AACTGCTGGCTGAAAAGGCAGTCTATGTGGGCGTCTGGATCCCAGCCCTC |
| | | CTCCTGACTATACCTGACTTCATCTTTGCCGACGTCAGCCAGGGGGACAT |
| | | CAGTCAGGGGGATGACAGGTACATCTGTGACCGCCTTTACCCCGATAGCC |
| | | TGTGGATGGTGGTGTTTCAATTCCAGCATATAATGGTGGGTCTCGTCCTG |
| | | CCCGGCATCGTCATCCTCTCCTGTTACTGCATCATCATCTCTAAGCTGTCA |
| | | CACTCCAAGGGCCACCAGAAGCGCAAGGCCCTCAAGACGACAGTCATCC |
| | | TCATCCTAGCTTTCTTTGCCTGCTGGCTGCCATATTATGTGGGGATCAGCA |
| | | TCGACTCCTTCATCCTTTTGGGGGTCATCAAGCAAGGATGTGACTTCGAG |
| | | AGCATCGTGCACAAGTGGATCTCCATCACAGAGGCCCTCGCCTTCTTCCA |
| | | CTGTTGCCTGAACCCCATCCTCTATGCCTTCCTCGGGGCCAAGTTCAAAA |
| | | GCTCTGCCCAGCATGCACTCAACTCCATGAGCAGAGGCTCCAGCCTCAAG |
| | | ATCCTTTCCAAAGGAAAGCGGGGTGGACACTCTTCCGTCTCCACGGAGTC |
| | | AGAATCCTCCAGTTTTCACTCCAGCTAA |
| 18 | human | CAGGTGCAGCTGCAGGAGAGCGGCCCCGGCCTGATCAAGCCCAGCCAGA |
| | CLDN18.2-BBZ-CXCR | CCCTGAGCCTGACCTGCACCGTGAGCGGCGGCAGCATCAGCAGCGGCTA |
| | 4 nucleotide sequence | CAACTGGCACTGGATCCGGCAGCCCCCCGGCAAGGGCCTGGAGTGGATC |
| | | GGCTACATCCACTACACCGGCAGCACCAACTACAACCCCGCCCTGCGGA |
| | | GCCGGGTGACCATCAGCGTGGACACCAGCAAGAACCAGTTCAGCCTGAA |
| | | GCTGAGCAGCGTGACCGCCGCCGACACCGCCATCTACTACTGCGCCCGG |
| | | ATCTACAACGGCAACAGCTTCCCCTACTGGGGCCAGGGCACCACCGTGA |
| | | CCGTGAGCAGCGGTGGAGGCGGTTCAGGCGGAGGTGGTTCTGGCGGTGG |
| | | CGGATCGGACATCGTGATGACCCAGAGCCCCGACAGCCTGGCCGTGAGC |
| | | CTGGGCGAGCGGGCCACCATCAACTGCAAGAGCAGCCAGAGCCTGTTCA |
| | | ACAGCGGCAACCAGAAGAACTACCTGACCTGGTACCAGCAGAAGCCCGG |
| | | CCAGCCCCCCAAGCTGCTGATCTACTGGGCCAGCACCCGGGAGAGCGGC |
| | | GTGCCCGACCGGTTCAGCGGCAGCGGCAGCGGCACCGACTTCACCCTGA |
| | | CCATCAGCAGCCTGCAGGCCGAGGACGTGGCCGTGTACTACTGCCAGAA |
| | | CGCCTACAGCTTCCCCTACACCTTCGGCGGCGGCACCAAGCTGGAGATCA |
| | | AGCGGACCACGACGCCAGCGCCGCGACCACCAACACCGGCGCCCACCAT |
| | | CGCGTCGCAGCCCCTGTCCCTGCGCCCAGAGGCGTGCCGGCCAGCGGCG |
| | | GGGGGCGCAGTGCACACGAGGGGGCTGGACTTCGCCTGTGATATCTACA |
| | | TCTGGGCGCCCTTGGCCGGGACTTGTGGGGTCCTTCTCCTGTCACTGGTTA |
| | | TCACCCTTTACTGCAAACGGGGCAGAAAGAAACTCCTGTATATATTCAAA |
| | | CAACCATTTATGAGACCAGTACAAACTACTCAAGAGGAAGATGGCTGTA |
| | | GCTGCCGATTTCCAGAAGAAGAAGAAGGAGGATGTGAACTGAGAGTGAA |
| | | GTTCAGCAGGAGCGCAGACGCCCCCGCGTACCAGCAGGGCCAGAACCAG |
| | | CTCTATAACGAGCTCAATCTAGGACGAAGAGAGGAGTACGATGTTTTGG |
| | | ACAAGAGACGTGGCCGGGACCCTGAGATGGGGGGAAAGCCGCAGAGAA |
| | | GGAAGAACCCTCAGGAAGGCCTGTACAATGAACTGCAGAAAGATAAGAT |
| | | GGCGGAGGCCTACAGTGAGATTGGGATGAAAGGCGAGCGCCGGAGGGG |
| | | CAAGGGGCACGATGGCCTTTACCAGGGTCTCAGTACAGCCACCAAGGAC |
| | | ACCTACGACGCCCTTCACATGCAGGCCCTGCCCCCTCGCGTGAAACAGAC |
| | | TTTGAATTTTGACCTTCTGAAGTTGGCAGGAGACGTTGAGTCCAACCCTG |
| | | GGCCCATGGAGGGGATCAGTATATACACTTCAGATAACTACACCGAGGA |
| | | AATGGGCTCAGGGGACTATGACTCCATGAAGGAACCCTGTTTCCGTGAA |
| | | GAAAATGCTAATTTCAATAAAATCTTCCTGCCCACCATCTACTCCATCAT |
| | | CTTCTTAACTGGCATTGTGGGCAATGGATTGGTCATCCTGGTCATGGGTT |
| | | ACCAGAAGAAACTGAGAAGCATGACGGACAAGTACAGGCTGCACCTGTC |
| | | AGTGGCCGACCTCCTCTTTGTCATCACGCTTCCCTTCTGGGCAGTTGATGC |
| | | CGTGGCAAACTGGTACTTTGGGAACTTCCTATGCAAGGCAGTCCATGTCA |
| | | TCTACACAGTCAACCTCTACAGCAGTGTCCTCATCCTGGCCTTCATCAGTC |
| | | TGGACCGCTACCTGGCCATCGTCCACGCCACCAACAGTCAGAGGCCAAG |
| | | GAAGCTGTTGGCTGAAAAGGTGGTCTATGTTGGCGTCTGGATCCCTGCCC |
| | | TCCTGCTGACTATTCCCGACTTCATCTTTGCCAACGTCAGTGAGGCAGAT |
| | | GACAGATATATCTGTGACCGCTTCTACCCCAATGACTTGTGGGTGGTTGT |
| | | GTTCCAGTTTCAGCACATCATGGTTGGCCTTATCCTGCCTGGTATTGTCAT |
| | | CCTGTCCTGCTATTGCATTATCATCTCCAAGCTGTCACACTCCAAGGGCC |
| | | ACCAGAAGCGCAAGGCCCTCAAGACCACAGTCATCCTCATCCTGGCTTTC |
| | | TTCGCCTGTTGGCTGCCTTACTACATTGGGATCAGCATCGACTCCTTCATC |
| | | CTCCTGGAAATCATCAAGCAAGGGTGTGAGTTTGAGAACACTGTGCACA |
| | | AGTGGATTTCCATCACCGAGGCCCTAGCTTTCTTCCACTGTTGTCTGAACC |
| | | CCATCCTCTATGCTTTCCTTGGAGCCAAATTTAAAACCTCTGCCCAGCAC |
| | | GCACTCACCTCTGTGAGCAGAGGGTCCAGCCTCAAGATCCTCTCCAAAGG |
| | | AAAGCGAGGTGGACATTCATCTGTTTCCACTGAGTCTGAGTCTTCAAGTT |
| | | TTCACTCCAGC |
| 19 | human CXCR4 amino | MEGISIYTSDNYTEEMGSGDYDSMKEPCFREENANFNKIFLPTIYSIIFLTGIVG |
| | acid sequence | NGLVILVMGYQKKLRSMTDKYRLHLSVADLLFVITLPFWAVDAVANWYFG |
| | | NFLCKAVHVIYTVNLYSSVLILAFISLDRYLAIVHATNSQRPRKLLAEKVVYV |
| | | GVWIPALLLTIPDFIFANVSEADDRYICDRFYPNDLWVVVFQFQHIMVGLILP |
| | | GIVILSCYCIIISKLSHSKGHQKRKALKTTVILILAFFACWLPYYIGISIDSFILLE |
| | | IIKQGCEFENTVHKWISITEALAFFHCCLNPILYAFLGAKFKTSAQHALTSVSR |
| | | GSSLKILSKGKRGGHSSVSTESESSSFHSS |
| 20 | CLDN18.2 full-length | MAVTACQGLGFVVSLIGIAGIIAATCMDQWSTQDLYNNPVTAVFNYQGLWR |
| | sequence | SCVRESSGFTECRGYFTLLGLPAMLQAVRALMIVGIVLGAIGLLVSIFALKCIR |
| | | IGSMEDSAKANMTLTSGIMFIVSGLCAIAGVSVFANMLVTNFWMSTANMYT |
| | | GMGGMVQTVQTRYTFGAALFVGWVAGGLTLIGGVMMCIACRGLAPEETN |
| | | YKAVSYHASGHSVAYKPGGFKASTGFGSNTKNKKIYDGGARTEDEVQSYPS |
| | | KHDYV |
| 21 | CLDN18.2-BBZ | QVQLQESGPGLIKPSQTLSLTCTVSGGSISSGYNWHWIRQPPGKGLEWIGYIH |
| | amino acid sequence | YTGSTNYNPALRSRVTISVDTSKNQFSLKLSSVTAADTAIYYCARIYNGNSFP |
| | | YWGQGTTVTVSSGGGGSGGGGSGGGGSDIVMTQSPDSLAVSLGERATINCK |
| | | SSQSLFNSGNQKNYLTWYQQKPGQPPKLLIYWASTRESGVPDRFSGSGSGTD |
| | | FTLTISSLQAEDVAVYYCQNAYSFPYTFGGGTKLEIKRTTTPAPRPPTPAPTIA |
| | | SQPLSLRPEACRPAAGGAVHTRGLDFACDIYIWAPLAGTCGVLLLSLVITLYC |
| | | KRGRKKLLYIFKQPFMRPVQTTQEEDGCSCRFPEEEEGGCELRVKFSRSADA |
| | | PAYQQGQNQLYNELNLGRREEYDVLDKRRGRDPEMGGKPQRRKNPQEGLY |
| | | NELQKDKMAEAYSEIGMKGERRRGKGHDGLYQGLSTATKDTYDALHMQA |
| | | LPPR |
| 22 | CLDN18.2-28Z | QVQLQESGPGLIKPSQTLSLTCTVSGGSISSGYNWHWIRQPPGKGLEWIGYIH |
| | amino acid sequence | YTGSTNYNPALRSRVTISVDTSKNQFSLKLSSVTAADTAIYYCARIYNGNSFP |
| | | YWGQGTTVTVSSGGGGSGGGGSGGGGSDIVMTQSPDSLAVSLGERATINCK |
| | | SSQSLFNSGNQKNYLTWYQQKPGQPPKLLIYWASTRESGVPDRFSGSGSGTD |
| | | FTLTISSLQAEDVAVYYCQNAYSFPYTFGGGTKLEIKRTTTPAPRPPTPAPTIA |
| | | SQPLSLRPEACRPAAGGAVHTRGLDFACDFWVLVVVGGVLACYSLLVTVAF |
| | | IIFWVRSKRSRLLHSDYMNMTPRRPGPTRKHYQPYAPPRDFAAYRSRVKFSR |
| | | SADAPAYQQGQNQLYNELNLGRREEYDVLDKRRGRDPEMGGKPQRRKNPQ |
| | | EGLYNELQKDKMAEAYSEIGMKGERRRGKGHDGLYQGLSTATKDTYDALH |
| | | MQALPPR |
| 23 | CLDN18.2-28BBZZ | QVQLQESGPGLIKPSQTLSLTCTVSGGSISSGYNWHWIRQPPGKGLEWIGYIH |
| | amino acid sequence | YTGSTNYNPALRSRVTISVDTSKNQFSLKLSSVTAADTAIYYCARIYNGNSFP |
| | | YWGQGTTVTVSSGGGGSGGGGSGGGGSDIVMTQSPDSLAVSLGERATINCK |
| | | SSQSLFNSGNQKNYLTWYQQKPGQPPKLLIYWASTRESGVPDRFSGSGSGTD |
| | | FTLTISSLQAEDVAVYYCQNAYSFPYTFGGGTKLEIKRTTTPAPRPPTPAPTIA |
| | | SQPLSLRPEACRPAAGGAVHTRGLDFACDFWVLVVVGGVLACYSLLVTVAF |
| | | IIFWVRSKRSRLLHSDYMNMTPRRPGPTRKHYQPYAPPRDFAAYRSKRGRK |
| | | KLLYIFKQPFMRPVQTTQEEDGCSCRFPEEEEGGCELRVKFSRSADAPAYQQ |
| | | GQNQLYNELNLGRREEYDVLDKRRGRDPEMGGKPQRRKNPQEGLYNELQK |
| | | DKMAEAYSEIGMKGERRRGKGHDGLYQGLSTATKDTYDALHMQALPPR |
| 24 | antiCLDN18.2-HCDR1 | SGYNWH |
| 25 | antiCLDN18.2-HCDR2 | YIHYTGSTNYNPALRS |
| 26 | antiCLDN18.2-HCDR3 | IYNGNSFPY |
| 27 | antiCLDN18.2-LCDR1 | KSSQSLFNSGNQKNYLT |
| 28 | antiCLDN18.2-LCDR2 | WASTRES |
| 29 | antiCLDN18.2-LCDR3 | QNAYSFPYT |

1. An immune effector cell for co-expressing a chemokine receptor, wherein the cell comprises: a receptor specifically recognizing claudin 18.2; and a protein recognizing SDF-1; preferably, the protein recognizing SDF-1 is an antibody recognizing SDF-1, or a receptor of SDF-1; more preferably, the receptor of SDF-1 is CXCR4 or CXCR7.

2. An immune effector cell for co-expressing a chemokine receptor, wherein the cell comprises: a receptor specifically recognizing a pancreatic cancer antigen; and a protein recognizing SDF-1; preferably, the protein recognizing SDF-1 is an antibody recognizing SDF-1, or a receptor of SDF-1; more preferably, the receptor of SDF-1 is CXCR4 or CXCR7.

3. The immune effector cell of claim 1, wherein the immune effector cell is selected from the group consisting of a T cell, a NK cell, a NKT cell, a macrophage, a CIK cell, and a stem cell-derived immune effector cell.

4. The immune effector cell of claim 1, wherein the receptor of SDF-1 is CXCR4, and preferably, an amino acid sequence of CXCR4 has at least 90% identity with the sequence shown in SEQ ID NO: 19.

5. The immune effector cell of claim 1, wherein the receptor specifically recognizing the claudin 18.2 or the receptor specifically recognizing the pancreatic cancer antigen is a chimeric receptor; preferably, the chimeric receptor is selected from the group consisting of: a chimeric antigen receptor (CAR), a chimeric T cell receptor, and a T cell antigen coupler (TAC).

6. The immune effector cell of claim 5, wherein: the chimeric receptor is a chimeric antigen receptor comprising an extracellular domain, a transmembrane domain and an intracellular signaling domain, which are connected in sequence; preferably, the extracellular domain of the chimeric antigen receptor comprises scFv, more preferably, the scFv comprising HDCR1, HCDR2, HCDR3 shown in SEQ ID NOs.: 24, 25, and 26, and LDCR1, LCDR2, LCDR3 shown in SEQ ID NOs.: 27, 28, and 29 respectively; more preferably, the extracellular domain of the chimeric antigen receptor comprises an amino acid sequence having at least 90% identity with the amino acid sequence shown in SEQ ID NO:2.

7. The immune effector cell of claim 4, wherein the immune effector cell comprises a nucleic acid having at least 90% identity with the nucleotide sequence shown in SEQ ID NO: 18.

8. The immune effector cell of claim 6, wherein the chimeric antigen receptor has at least 90% identity with the amino acid sequence shown in SEQ ID NOs.: 21, 22 or 23.

9. An expression construct comprising the expression substance of a receptor blinding tumor-associated antigen and the expression substance of a protein recognizing SDF-1, which are connected in sequence; preferably, the two substances are connected with a tandem fragment; more preferably, the tandem fragment comprising F2A, PA2, T2A, and/or E2A.

10. The expression construct of claim 9, wherein the protein recognizing SDF-1 is an antibody recognizing SDF-1, or a receptor of SDF-1; more preferably, the receptor is CXCR4 or CXCR7.

11. The expression construct of claim 10, wherein the receptor of SDF-1 is CXCR4; preferably, the nucleic acid sequence of expression substance of CXCR4 has at least 90% identity with the nucleic acid sequence encoding the amino acid sequence shown in SEQ ID NO: 19.

12. The expression construct of claim 9, wherein the receptor binding tumor-associated antigen has at least 90% identity with the amino acid sequence shown in SEQ ID NO: 21, 22 or 23.

13-18. (canceled)


SEQUENCE LISTING (29 sequences)

SEQ ID NO: 1 - DNA, 741 bp, Artificial Sequence
  CLDN18.2-scFv nucleotide sequence
caggtgcagctgcaggagagcggccccggcctgatcaagcccagccagaccctgagcctgacctgcaccgtgagcggcggcagcatcagcagcggctacaactggcactggatccggcagccccccggcaagggcctggagtggatcggctacatccactacaccggcagcaccaactacaaccccgccctgcggagccgggtgaccatcagcgtggacaccagcaagaaccagttcagcctgaagctgagcagcgtgaccgccgccgacaccgccatctactactgcgcccggatctacaacggcaacagcttcccctactggggccagggcaccaccgtgaccgtgagcagcggtggaggcggttcaggcggaggtggttctggcggtggcggatcggacatcgtgatgacccagagccccgacagcctggccgtgagcctgggcgagcgggccaccatcaactgcaagagcagccagagcctgttcaacagcggcaaccagaagaactacctgacctggtaccagcagaagcccggccagccccccaagctgctgatctactgggccagcacccgggagagcggcgtgcccgaccggttcagcggcagcggcagcggcaccgacttcaccctgaccatcagcagcctgcaggccgaggacgtggccgtgtactactgccagaacgcctacagcttcccctacaccttcggcggcggcaccaagctggagatcaagcgg

SEQ ID NO: 2 - protein, 247 aa, Artificial Sequence
  CLDN18.2-scFv amino acid sequence
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKR

SEQ ID NO: 3 - DNA, 81 bp, Artificial Sequence
  mouse CD8alpha signal peptide nucleotide sequence
atggcctcaccgttgacccgctttctgtcgctgaacctgctgctgctgggtgagtcgattatcctggggagtggagaagct

SEQ ID NO: 4 - protein, 27 aa, Artificial Sequence
  mouse CD8alpha signal peptide amino acid sequence
MASPLTRFLSLNLLLLGESIILGSGEA

SEQ ID NO: 5 - DNA, 216 bp, Artificial Sequence
  Mouse CD8 hinge and transmembrane domain nucleotide sequence
actactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctaccacaggagccga

SEQ ID NO: 6 - protein, 72 aa, Artificial Sequence
  Mouse CD8 hinge and transmembrane domain  amino acid sequence
TTTKPVLRTPSPVHPTGTSQPQRPEDCRPRGSVKGTGLDFACDIYIWAPLAGICVALLLSLIITLICYHRSR

SEQ ID NO: 7 - DNA, 135 bp, Artificial Sequence
  Mouse 4-1BB intracellular signaling domain nucleotide sequence
aaatggatcaggaaaaaattcccccacatattcaagcaaccatttaagaagaccactggagcagctcaagaggaagatgcttgtagctgccgatgtccacaggaagaagaaggaggaggaggaggctatgagctg

SEQ ID NO: 8 - protein, 45 aa, Artificial Sequence
  Mouse 4-1BB intracellular signaling domain amino acid sequence
KWIRKKFPHIFKQPFKKTTGAAQEEDACSCRCPQEEEGGGGGYEL

SEQ ID NO: 9 - DNA, 321 bp, Artificial Sequence
  nucleotide sequence of CD3zeta, the intracellular segment of mouse CD3
agcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcc

SEQ ID NO: 10 - protein, 107 aa, Artificial Sequence
  amino acid sequence of CD3zeta, the intracellular segment of mouse CD3
SRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLA

SEQ ID NO: 11 - DNA, 66 bp, Artificial Sequence
  F2A  nucleotide sequence
gtgaaacagactttgaattttgaccttctgaagttggcaggagacgttgagtccaaccctgggccc

SEQ ID NO: 12 - protein, 22 aa, Artificial Sequence
  F2A  amino acid sequence
VKQTLNFDLLKLAGDVESNPGP

SEQ ID NO: 13 - DNA, 1074 bp, Artificial Sequence
  mouse CXCR4  nucleotide sequence
atggaaccgatcagtatatacacttctgataactactctgaagaagtgggttctggagactatgactccaacaaggaaccctgcttccgggatgaaaacgtccatttcaataggatcttcctgcccaccatctacttcatcatcttcttgactggcatagtcggcaatggattggtgatcctggtcatgggttaccagaagaagctaaggagcatgacggacaagtaccggctgcacctgtcagtggctgacctcctctttgtcatcacactccccttctgggcagttgatgccatggctgactggtactttgggaaatttttgtgtaaggctgtccatatcatctacactgtcaacctctacagcagcgttctcatcctggccttcatcagcctggaccggtacctcgctattgtccacgccaccaacagtcagaggccaaggaaactgctggctgaaaaggcagtctatgtgggcgtctggatcccagccctcctcctgactatacctgacttcatctttgccgacgtcagccagggggacatcagtcagggggatgacaggtacatctgtgaccgcctttaccccgatagcctgtggatggtggtgtttcaattccagcatataatggtgggtctcgtcctgcccggcatcgtcatcctctcctgttactgcatcatcatctctaagctgtcacactccaagggccaccagaagcgcaaggccctcaagacgacagtcatcctcatcctagctttctttgcctgctggctgccatattatgtggggatcagcatcgactccttcatccttttgggggtcatcaagcaaggatgtgacttcgagagcatcgtgcacaagtggatctccatcacagaggccctcgccttcttccactgttgcctgaaccccatcctctatgccttcctcggggccaagttcaaaagctctgcccagcatgcactcaactccatgagcagaggctccagcctcaagatcctttccaaaggaaagcggggtggacactcttccgtctccacggagtcagaatcctccagttttcactccagctaa

SEQ ID NO: 14 - protein, 357 aa, Artificial Sequence
  mouse CXCR4  amino acid sequence
MEPISIYTSDNYSEEVGSGDYDSNKEPCFRDENVHFNRIFLPTIYFIIFLTGIVGNGLVILVMGYQKKLRSMTDKYRLHLSVADLLFVITLPFWAVDAMADWYFGKFLCKAVHIIYTVNLYSSVLILAFISLDRYLAIVHATNSQRPRKLLAEKAVYVGVWIPALLLTIPDFIFADVSQGDISQGDDRYICDRLYPDSLWMVVFQFQHIMVGLVLPGIVILSCYCIIISKLSHSKGHQKRKALKTTVILILAFFACWLPYYVGISIDSFILLGVIKQGCDFESIVHKWISITEALAFFHCCLNPILYAFLGAKFKSSAQHALNSMSRGSSLKILSKGKRGGHSSVSTESESSSFHSS

SEQ ID NO: 15 - DNA, 1413 bp, Artificial Sequence
  CLDN18.2- BBZ  nucleotide sequence
caggtgcagctgcaggagagcggccccggcctgatcaagcccagccagaccctgagcctgacctgcaccgtgagcggcggcagcatcagcagcggctacaactggcactggatccggcagccccccggcaagggcctggagtggatcggctacatccactacaccggcagcaccaactacaaccccgccctgcggagccgggtgaccatcagcgtggacaccagcaagaaccagttcagcctgaagctgagcagcgtgaccgccgccgacaccgccatctactactgcgcccggatctacaacggcaacagcttcccctactggggccagggcaccaccgtgaccgtgagcagcggtggaggcggttcaggcggaggtggttctggcggtggcggatcggacatcgtgatgacccagagccccgacagcctggccgtgagcctgggcgagcgggccaccatcaactgcaagagcagccagagcctgttcaacagcggcaaccagaagaactacctgacctggtaccagcagaagcccggccagccccccaagctgctgatctactgggccagcacccgggagagcggcgtgcccgaccggttcagcggcagcggcagcggcaccgacttcaccctgaccatcagcagcctgcaggccgaggacgtggccgtgtactactgccagaacgcctacagcttcccctacaccttcggcggcggcaccaagctggagatcaagcggactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctaccacaggagccgaaaatggatcaggaaaaaattcccccacatattcaagcaaccatttaagaagaccactggagcagctcaagaggaagatgcttgtagctgccgatgtccacaggaagaagaaggaggaggaggaggctatgagctgagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcc

SEQ ID NO: 16 - protein, 471 aa, Artificial Sequence
  CLDN18.2- BBZ  amino acid sequence
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 17 - DNA, 2553 bp, Artificial Sequence
  CLDN18.2-BBZ-CXCR4  nucleotide sequence
caggtgcagctgcaggagagcggccccggcctgatcaagcccagccagaccctgagcctgacctgcaccgtgagcggcggcagcatcagcagcggctacaactggcactggatccggcagccccccggcaagggcctggagtggatcggctacatccactacaccggcagcaccaactacaaccccgccctgcggagccgggtgaccatcagcgtggacaccagcaagaaccagttcagcctgaagctgagcagcgtgaccgccgccgacaccgccatctactactgcgcccggatctacaacggcaacagcttcccctactggggccagggcaccaccgtgaccgtgagcagcggtggaggcggttcaggcggaggtggttctggcggtggcggatcggacatcgtgatgacccagagccccgacagcctggccgtgagcctgggcgagcgggccaccatcaactgcaagagcagccagagcctgttcaacagcggcaaccagaagaactacctgacctggtaccagcagaagcccggccagccccccaagctgctgatctactgggccagcacccgggagagcggcgtgcccgaccggttcagcggcagcggcagcggcaccgacttcaccctgaccatcagcagcctgcaggccgaggacgtggccgtgtactactgccagaacgcctacagcttcccctacaccttcggcggcggcaccaagctggagatcaagcggactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctaccacaggagccgaaaatggatcaggaaaaaattcccccacatattcaagcaaccatttaagaagaccactggagcagctcaagaggaagatgcttgtagctgccgatgtccacaggaagaagaaggaggaggaggaggctatgagctgagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggccgtgaaacagactttgaattttgaccttctgaagttggcaggagacgttgagtccaaccctgggcccatggaaccgatcagtatatacacttctgataactactctgaagaagtgggttctggagactatgactccaacaaggaaccctgcttccgggatgaaaacgtccatttcaataggatcttcctgcccaccatctacttcatcatcttcttgactggcatagtcggcaatggattggtgatcctggtcatgggttaccagaagaagctaaggagcatgacggacaagtaccggctgcacctgtcagtggctgacctcctctttgtcatcacactccccttctgggcagttgatgccatggctgactggtactttgggaaatttttgtgtaaggctgtccatatcatctacactgtcaacctctacagcagcgttctcatcctggccttcatcagcctggaccggtacctcgctattgtccacgccaccaacagtcagaggccaaggaaactgctggctgaaaaggcagtctatgtgggcgtctggatcccagccctcctcctgactatacctgacttcatctttgccgacgtcagccagggggacatcagtcagggggatgacaggtacatctgtgaccgcctttaccccgatagcctgtggatggtggtgtttcaattccagcatataatggtgggtctcgtcctgcccggcatcgtcatcctctcctgttactgcatcatcatctctaagctgtcacactccaagggccaccagaagcgcaaggccctcaagacgacagtcatcctcatcctagctttctttgcctgctggctgccatattatgtggggatcagcatcgactccttcatccttttgggggtcatcaagcaaggatgtgacttcgagagcatcgtgcacaagtggatctccatcacagaggccctcgccttcttccactgttgcctgaaccccatcctctatgccttcctcggggccaagttcaaaagctctgcccagcatgcactcaactccatgagcagaggctccagcctcaagatcctttccaaaggaaagcggggtggacactcttccgtctccacggagtcagaatcctccagttttcactccagctaa

SEQ ID NO: 18 - DNA, 2535 bp, Artificial Sequence
  human CLDN18.2-BBZ-CXCR4  nucleotide sequence
caggtgcagctgcaggagagcggccccggcctgatcaagcccagccagaccctgagcctgacctgcaccgtgagcggcggcagcatcagcagcggctacaactggcactggatccggcagccccccggcaagggcctggagtggatcggctacatccactacaccggcagcaccaactacaaccccgccctgcggagccgggtgaccatcagcgtggacaccagcaagaaccagttcagcctgaagctgagcagcgtgaccgccgccgacaccgccatctactactgcgcccggatctacaacggcaacagcttcccctactggggccagggcaccaccgtgaccgtgagcagcggtggaggcggttcaggcggaggtggttctggcggtggcggatcggacatcgtgatgacccagagccccgacagcctggccgtgagcctgggcgagcgggccaccatcaactgcaagagcagccagagcctgttcaacagcggcaaccagaagaactacctgacctggtaccagcagaagcccggccagccccccaagctgctgatctactgggccagcacccgggagagcggcgtgcccgaccggttcagcggcagcggcagcggcaccgacttcaccctgaccatcagcagcctgcaggccgaggacgtggccgtgtactactgccagaacgcctacagcttcccctacaccttcggcggcggcaccaagctggagatcaagcggaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgtgaaacagactttgaattttgaccttctgaagttggcaggagacgttgagtccaaccctgggcccatggaggggatcagtatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagc

SEQ ID NO: 19 - protein, 352 aa, Artificial Sequence
  human CXCR4 amino acid sequence
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 20 - protein, 261 aa, Artificial Sequence
  CLDN18.2 full-length sequence
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 21 - protein, 471 aa, Artificial Sequence; identical to SEQ ID NO: 16
  CLDN18.2- BBZ  amino acid sequence

SEQ ID NO: 22 - protein, 473 aa, Artificial Sequence
  CLDN18.2- 28Z  amino acid sequence
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 23 - protein, 515 aa, Artificial Sequence
  CLDN18.2- 28BBZZ  amino acid sequence
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 24 - protein, 6 aa, Artificial Sequence
  antiCLDN18.2-HCDR1
SGYNWH

SEQ ID NO: 25 - protein, 16 aa, Artificial Sequence
  antiCLDN18.2-HCDR2
YIHYTGSTNYNPALRS

SEQ ID NO: 26 - protein, 9 aa, Artificial Sequence
  antiCLDN18.2-HCDR3
IYNGNSFPY

SEQ ID NO: 27 - protein, 17 aa, Artificial Sequence
  antiCLDN18.2-LCDR1
KSSQSLFNSGNQKNYLT

SEQ ID NO: 28 - protein, 7 aa, Artificial Sequence
  antiCLDN18.2-LCDR2
WASTRES

SEQ ID NO: 29 - protein, 9 aa, Artificial Sequence
  antiCLDN18.2-LCDR3
QNAYSFPYT